(19) United States
(12) Patent Application Publication
(10) Pub. No.: US 2009/0054854 A1
(43) Pub. Date: Feb. 26, 2009

(54) MICRODIALYSIS CATHETER AND PROCESS FOR MANUFACTURE

(76) Inventors: Gernot Hochmuth, Mannheim (DE); Reinhold Mischler, Ludwigshafen (DE)

Correspondence Address: ROCHE DIAGNOSTICS OPERATIONS INC., 9115 Hague Road, Indianapolis, IN, 46250-0457, US

(21) Appl. No.: 12/196,018

(22) Filed: Aug. 21, 2008

(30) Foreign Application Priority Data
Aug. 24, 2007 (EP) 07114968.6

(51) Int. Cl. A61L 29/16; A61M 25/00

(52) U.S. Cl. 604/265; 427/2.3

(57) ABSTRACT

A microdialysis catheter for partial implantation is used to supply a perfusion fluid and remove a dialysate, such as glucose, and comprises a carrier, at least one opening in the carrier, and a non-porous coating over the opening. The carrier contains at least one channel for supplying a perfusion fluid and for removing a dialysate and an area intended for implantation. The opening on the carrier has a connection with the channel for the passage of fluid. The non-porous coating is externally bonded to the carrier. The non-poroous coating is diffusion-permeable to an analyte and covers the area intended for implantation and the opening. A process for manufacturing the microdialysis catheter is also disclosed.

### REFERENCE

This application is based on and claims priority to European Patent Application No. 07114968.6 filed Aug. 24, 2007, which is hereby incorporated by reference.

### FIELD

The disclosure relates to a process for the manufacture of a microdialysis catheter and to a microdialysis catheter. Microdialysis catheters of this kind are generally known and are used to measure certain parameters within biological tissue (for example inside the human body) by means of microdialysis. An example of such a parameter is the level of blood glucose in subcutaneous tissue.

### BACKGROUND

U.S. Pat. No. 6,572,566 B2 relates to a system for determining the concentration of at least one analyte in a bodily fluid. The system contains a canal with an exchange area via which substances can be taken up from the surrounding bodily fluid; it also has, downstream from the exchange area, a sensor with which the concentration of an analyte can be ascertained. The system has at least one integrated reservoir which is connected to the canal. The dialysis area is formed by the canal which is open at the top and covered by a membrane or a perforated area. If perfusion fluid passes through the canal while the exchange area is in contact with a bodily fluid, the perfusion fluid takes up substances from the bodily fluid. The membrane or the perforated structure is glued or sealed onto the dialysis area. Covering a canal structure with known membrane materials while maintaining the porous properties of the membrane is however difficult.

U.S. Pat. No. 6,632,315 B2 concerns a process for the manufacture of a microdialysis catheter with the following steps: extrusion of a flexible material to form an elongate catheter body with an essentially cylindrical outer surface and a large number of continuous internal canals running in the longitudinal direction of the catheter body; sealing a free end of the catheter body; providing an opening in the surface at a distance from the free end which extends into the catheter body and communicates with part of a first of the canals; providing a communicating opening between the first canal and a second of the canals at a distance from the free end; and, covering the opening with a microdialysis membrane. The microdialysis membrane is pushed onto the catheter body via the opening.

The subject of U.S. Pat. No. 4,694,832 is a dialysis probe comprising a dialysis membrane and ducts for the flow of perfusion fluid through the membrane. The dialysis membrane is surrounded by a casing which supports and partly exposes the membrane and is moreover stiffer than the membrane itself. The ducts form diametrically opposed canals in the catheter which end within the tubular membrane. The relatively stiff casing is intended to prevent the biological tissue from compressing the tubular membrane when the catheter is inserted into it and thereby impairing its function and flow within the tubular membrane. The addition of the supportive casing makes this known catheter resource-intensive and costly to manufacture, and it has to have a relatively large diameter since the membrane has to be inserted into the casing.

European Patent No. EP 0 973 573 B1 relates to a catheter for measuring chemical parameters, in particular for insertion into biological tissue, into fluids or the like. The catheter contains a return canal and a tubular semi-permeable membrane. There are spacers in the form of ribs which keep the inside wall of the tubular membrane away from the outside wall of a tubular part arranged inside the tubular membrane. However, the spacers support only parts of the membrane and are not connected to the membrane or are connected to it only at a small number of places, so that many areas of the membrane are vulnerable to mechanical forces from outside.

International Patent Application No. WO 03/020352 A2 concerns a catheter with an essentially U-shaped bent hollow-fiber membrane with two legs and a bent area; the hollow-fiber membrane is heat-treated and/or treated with at least one solvent, at least over its bent area, so as to achieve as small as possible an overall diameter of the catheter in the patent lumen of the hollow-fiber membrane. The hollow-fiber membrane can be reinforced; the reinforcement can be a wire, a thread and/or a cord which is connected, glued or welded to the two legs. With this solution too, the relatively large areas of the hollow-fiber membrane that are not directly connected with the reinforcing structure are vulnerable to mechanical forces from outside.

The disclosure is based on the task of developing a process for the manufacture of a microdialysis catheter and supplying a microdialysis catheter which avoid the disadvantages of the existing state of the art. In particular, the task of embodiments of the present invention is to supply a microdialysis catheter which is uncomplicated and cheap to manufacture and use and is sufficiently miniaturizable that it leaves as small a wound as possible on implantation. The catheter should also be sufficiently mechanically stable to minimize the risk of parts of the catheter being left in the tissue on explantation.

### SUMMARY

The task is solved according to the teachings of the disclosure by means of a process for the manufacture of a microdialysis catheter comprising the steps: provision of a carrier containing at least one channel for the supply of a perfusion fluid and for the removal of a dialysate and having an area intended for implantation containing at least one opening that has a connection with the channel for the passage of fluid; and, coating of the carrier with a non-porous coating which is diffusion-permeable to an analyte, externally at least partly covers the area intended for implantation, is firmly bonded to the carrier and covers the opening.

The task is also solved according to the teachings of the disclosure by a microdialysis catheter which is preferably manufactured by a process according to the teachings of the disclosure and which comprises a carrier containing at least one channel for supplying a perfusion fluid and for removing a dialysate and having an area intended for implantation, containing at least one opening that has a connection with the channel for the passage of fluid, where the carrier has a non-porous coating which is diffusion-permeable to an analyte, which externally at least partly covers the area intended for implantation, is firmly bonded to the carrier and covers the at least one opening.

The microdialysis catheter according to the disclosure is manufactured using a carrier which gives the catheter mechanical stability. The carrier can for example take the form of a tube or a flat carrier substrate with a cover. This is preferably a carrier made of polyurethane (PUR), polyamide (PA), polyethylene terephthalate (PET) or stainless steel. These materials are advantageous as regards their biocompatibility, workability and/or availability. If tubes or pipes are used as carriers, these should preferably have an external diameter of 0.5 to 0.75 mm. If a flat carrier substrate is used, a flat carrier substrate can have a thickness of 0.25 to 0.5 mm (plus the thickness of the covering provided by existing coatings) and a width of 0.5 to 0.75 mm.

Inside the carrier there should be at least one channel for supplying a perfusion fluid and for removing a dialysate. The channels can for example be largely separate lumina of a tubular carrier, inner tubes running inside the carrier or any other form of canal structure. One variant embodiment makes provision for a U-shaped, bent tubular channel in the carrier. Another variant embodiment makes provision for two tubular channels running parallel to one another which are connected to one another for the exchange of fluid via openings in the region of their distal ends. The distal ends are the ends which are arranged in or close to an area of the microdialysis catheter which is intended for implantation. The area intended for implantation is a section of the carrier which is situated in the biological tissue when the catheter is implanted. The tubular channels in the carrier can for example be made of plastic or metal.

The area of the carrier which is intended for implantation has an opening into the area surrounding the catheter which is linked to the at least one channel for the exchange of fluid. Through this opening an analyte can for example pass from a bodily fluid in the surroundings of the implanted area into the inside of the carrier and into a fluid present in the at least one channel. The dialysate obtained in this way is removed via a channel and can then be tested for the analyte by means of a sensor, for example.

The diameter of the opening in the carrier can be between about 1 μm to about 10 μm. The area of the opening can be about 1 μm² to about 100 μm².

The at least one opening in the carrier is, according to the disclosure, closed by a non-porous coating which is diffusion-permeable to an analyte, externally at least partly covers the area intended for implantation, is firmly bonded to the carrier and covers the at least one opening. The coating can be a membrane with a thickness of about 1 μm to about 100 μm. The coating is diffusion-permeable to the analyte (for example glucose) and is not porous. Non-porous in this context means that there are no microscopically visible pores in the coating. There is generally also a difference from porous membranes in that the non-porous coating may have a three-dimensional polymer network which swells considerably in an aqueous solution so that the volume in which the analyte can move by diffusion can be considerably enlarged. By contrast, the pores in a porous membrane are generally fully formed in size and shape even in the dry state. Accordingly, the polymer network of the porous membrane swells only slightly in aqueous solution, so that the pore volume present in the dry state is more or less the same as the pore volume when wet by the aqueous solution. For further differences between non-porous, i.e. dense membranes and porous, in particular microporous membranes and substance-transporting mechanisms in both types of material, reference can for example be made to U.S. Pat. No. 5,428,123.

The microdialysis membrane materials used in the state of the art have, unlike the coating used according to the disclosure, a quantifiable microscopic porosity. The transport of the analyte through such microporous membranes from the tissue surrounding the catheter into the inner lumen of the catheter occurs by diffusion through the pores in the membrane which are wet with aqueous solution. By contrast, the coating used in embodiment of the present invention is a film which is permeable to an analyte and the permeability of which is not defined by microscopic porosity.

The coating can be a polymer which is capable of taking up small molecules (such as glucose) with water and allowing them to pass and of holding back larger molecules because of the entwined polymer structure. The coating can be a hydrophilic polymer or consists of a hydrophilic polymer. For example, a coating of hydrophilic polyurethane can be manufactured on the carrier in the area intended for implantation. The transport of an analyte through the coating occurs by diffusion through the aqueous solution taken up in the three-dimensional polymer network of the membrane from the surroundings of the carrier. The three-dimensional network of the polymer chains is swollen but not dissolved by the aqueous solution.

The coating used as membrane can cover the carrier in a part of the area intended for implantation, in the whole of the area intended for implantation, can extend beyond the area intended for implantation or can cover the entire carrier and be firmly bonded to it. According to the disclosure, the material for coating the carrier is deposited directly onto the carrier. The permeable film is therefore created on the carrier and is intimately linked (firmly bonded) to it on the entire surface of the carrier covered by the film.

The microdialysis catheter according to embodiments of the invention has the advantage that it encloses the relatively stable carrier, which has openings only at defined places, so that the mechanical resilience of the catheter is great. The coating used as membrane is (apart from in the region of the openings) bound to the substrate over its entire area, so that there is a secure bond. The coating covers the defined openings in the carrier unsupported. The unsupported area of the coating is therefore reduced to the areas of what are generally very small openings, thus minimizing the risk of damage to the membrane.

No complex assembly stages are needed in the manufacture of the microdialysis catheter according to embodiments of the invention, so that the catheter can readily be manufactured in miniaturized form. The manufacture of the channels in the carrier and the necessary connections between the channels and the outside area can be done using common processes that are the state of the art. The production of the coating (membrane production process) is preferably based on controllable self-organizing mechanisms. For example, the carrier can be dipped in a polymer solution and the polymer can then be left to harden. The parameters relevant for these processes are surface tension and the viscosity of the polymer solution.

According to embodiments of the present invention the carrier is therefore coated with a polymer solution in the area intended for implantation, to make the coating. The polymer solution used is a solution in which a polymer is present dissolved in a solvent. According to a preferred variant the carrier is coated with a solution of hydrophilic polyurethane HPU in ethanol to make the coating. The coating covers and, by virtue of the self-organizing processes in creation of the layer (because of the viscosity and/or the surface tension), covers the carrier or the openings in the carrier.

To make the coating, the carrier is preferably dipped in a polymer solution or is sprayed with a polymer solution, or the coating is created by squeegeeing or by rotary coating with a polymer solution. Dipping of the carrier or the spraying of the polymer solution is an option available for carriers of any desired shapes. Squeegeeing or rotary coating (spin coating) can in particular be used to create coatings on planar carriers.

The carrier can be coated with a polymer solution which can then be hardened, in particular one which is hardened by heating, irradiation with electromagnetic radiation or by contact with water. The polymer membrane layer can be produced by local evaporation of the solvent in which the polymer is dissolved. In addition, a hardening reaction of the polymer initiated by irradiation with light of a suitable wavelength, thermal action or by contact with atmospheric humidity is for example possible.

According to embodiments of the present invention, the coating is covered with a layer (covering layer) of a biocompatible (non-porous or porous) material. The coating is therefore coated with a further biocompatibility-improving layer through which the analyte can flow.

With a non-porous layer of the biocompatible material, substance transport (as with the underlying coating) occurs by diffusion. One aspect with a non-porous biocompatible layer is a short diffusion time of the analyte through the layer. The diffusion time is substantially influenced by the layer thickness. The biocompatible layer should therefore be applied as thinly as possible. The thickness of the biocompatible layer should preferably be between about 1 μm and about 10 μm. The covering layer to improve biocompatibility can for example be produced using 2-methacryloyloxyethyl phosphorylcholine polymer (MPC) which has no microscopic porosity.

Substance transport through a porous layer of the biocompatible material is generally based on diffusion of the analyte within an aqueous solution which wets the pores and usually also fills them. Consequently, the speed at which the analyte passes through the biocompatible layer is defined by a diffusion constant. For this reason, having a thin biocompatible layer is desirable for the speed of passage; its thickness can be between about 1 μm and about 10 μm. The fluid can thus pass through the porous layer relatively quickly and reach the coating which is diffusion-permeable to the analyte.

According to embodiments of the present invention, the carrier of the microdialysis catheter is a tube the distal end of which is sealed and has the at least one side opening covered by a non-porous coating diffusion-permeable to an analyte, where the tube contains a longitudinal inner space containing at least one fluid channel for supplying a perfusion fluid and for removing a dialysate, where the fluid channel has a connection with the side opening for the passage of fluid. The area intended for implantation encloses the distal end of the tube. The at least one side opening allows the passage of an analyte-containing fluid from the surroundings of the catheter through the coating into the lumen of the tube and from there into the fluid channel for removal of the dialysate which has a connection with the side opening for the passage of fluid. The fluid channels in the inner space of the tube are preferably two lumina separated by a dividing wall or two inner tubes with a connection for the passage of fluid between them via at least one opening.

One possible variant of a tubular carrier with fluid channels is a multi-lumen tube. The outside wall of a multi-lumen tube, i.e. a tube in which several (such as two) lumina with passage of fluid between them prevented by dividing walls were created, has openings on the side in the area which is inside the body when the microdialysis catheter is in use, making possible a connection for the passage of fluid from the surrounding outer space to the inner space of the microdialysis catheter. The dividing wall between two lumina of the multi-lumen tube is structured in such a way shortly before the distal end of the microdialysis catheter that the two lumina have a connection for the passage of fluid between them. The dividing wall can, for example, starting from the front surface of the multi-lumen tube, be notched or removed using a suitable tool (such as a laser). At least one of the fluid-linked lumina is used to supply a perfusion fluid. At least one more of the fluid-linked lumina is used to remove the dialysate. The multi-lumen tube structured in this way has a seal at the distal end to prevent the passage of fluid from the surrounding outer area in that, for example, the openings at the ends of the lumina are sealed with a sealing compound. The tube with the side openings and the connection for the passage of fluid between the lumina and the seal of the distal end receives a non-porous outer coating which is diffusion-permeable to the analyte. The coating preferably covers the outside wall of the tube completely in a defined area which, when the catheter is implanted, is in the tissue. The coating is connected with the outside wall. The side openings created are covered by the coating so that the lumina can be reached by the analyte. The multi-lumen tube with the said coating if necessary receives a further biocompatible coating through which the analyte can flow.

According to further embodiments of the present invention, the carrier of the microdialysis catheter is a flat substrate with a cover, where the substrate has, as a channel, a canal structure for supplying a perfusion fluid and for removing a dialysate, where the canal structure is closed by the cover of the flat substrate, and where the substrate or the cover has at least one opening which is covered by a non-porous coating which is diffusion-permeable to an analyte and is connected with the canal structure for the passage of fluid. In the manufacture of such a microdialysis catheter, a canal structure allowing the supply of a perfusion fluid and the removal of the dialysate is introduced into a flat substrate. The canal structure is closed on all sides, for example by covering a groove in the surface of the substrate with a cover plate or cover foil. Openings are made into the substrate and/or into the cover plate/foil from outside which allow a connection for the passage of fluid to be made between the outer area surrounding the microdialysis catheter and the canal structure. The substrate with the openings in it and/or the cover with the openings receives an outer non-porous coating which is diffusion-permeable to the analyte. The coating covers the outside wall of the substrate and the cover. The coating covers the created openings. The substrate with the aforementioned coating is if necessary given a further biocompatible coating through which the analyte can flow.

### BRIEF DESCRIPTION OF THE DRAWINGS

Embodiments of the invention are explained in greater detail below using the drawings:

FIG. 1 shows a schematic view of a first embodiment of a microdialysis catheter with a tubular carrier and two inner tubes as fluid channels;

FIG. 2 shows a schematic view of a second embodiment of a microdialysis catheter with a tubular carrier and two inner tubes as fluid channels;

FIGS. 3.1 to 3.3 show a schematic view of a third embodiment of a microdialysis catheter with a carrier in the form of a multi-lumen tube; and,

FIGS. 4.1 to 4.3 show a schematic view of a fourth embodiment of a microdialysis catheter with a carrier in the form of a flat substrate.

### DETAILED DESCRIPTION

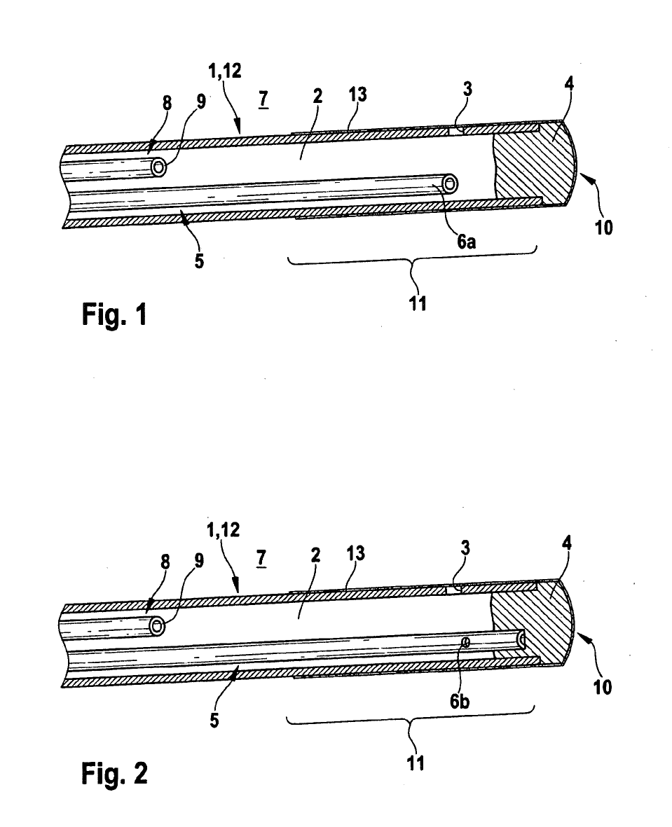

FIG. 1 shows schematically a section from a microdialysis catheter according to an embodiment of the invention which was manufactured using the disclosed process for manufacturing the microdialysis catheter.

The microdialysis catheter comprises a carrier 1 in the form of a tube 12. The longitudinal inner space 2 of the tube 12 contains two inner tubes 5 and 8 as fluid channels. The first inner tube 5 is intended for the supply of a perfusion fluid and the second inner tube 8 for the removal of a dialysate. The inner tubes 5 and 8 can for example be made from a polymer or a metal. The distal end 10 of tube 12 is closed by a seal 4 and thus has a seal (for example in the form of a sealing compound or a cover) to prevent the passage of fluid from the surrounding outer space 7.

The carrier 1 has an area 11 intended for implantation. In this area 11 the tube 12 has at least one side opening 3 which has a connection for the passage of fluid, via the inner space 2, with the two inner tubes 5 and 8 via its open ends 6a and 9. The first inner tube 5 sticks further into the tube 12 than the second inner tube 8, and its open end 6a is therefore positioned closer to the seal 4 than the open end 9 of the second inner tube 8. The flow direction of a perfusate introduced at the proximal end (not shown) of the first fluid channel (first inner tube 5) after it has passed out of the open end 6a into the inner space 2, therefore undergoes, before the seal 4 at the distal end 10 of the microdialysis catheter, a change in direction, flows past the opening 3 and passes through the open end 9 of the second fluid channel 8 and into that channel.

Carrier 1 has a non-porous coating 13 which is diffusion-permeable to an analyte and covers the area 11 intended for implantation externally and is firmly bonded to carrier 1. The coating 13 covers the at least one opening 3.

FIG. 2 shows schematically a section from a further microdialysis catheter according to an embodiment of the invention which was manufactured using the process according to an embodiment of the invention.

The catheter according to FIG. 2 has essentially the same structure as the catheter according to FIG. 1; the same reference numbers denote the same components. In this embodiment the first inner tube 5 is designed so that it extends into the sealing compound of the seal 4 and its end is sealed by it. The first inner tube 5 also has a side opening 6b close to the distal end 10 through which the perfusion fluid gets into the inner space in the tube 12.

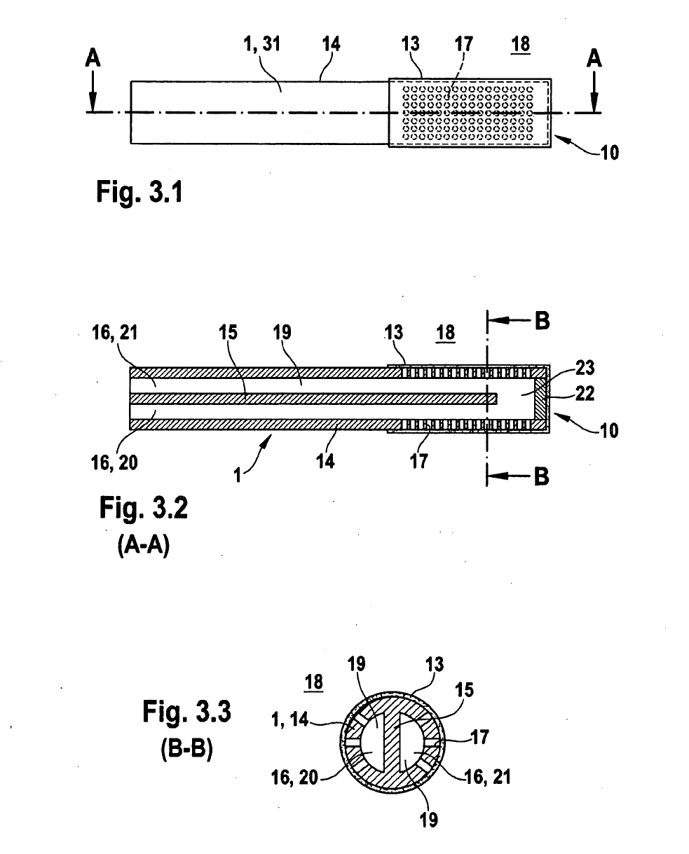

FIGS. 3.1 to 3.3 show schematically a third embodiment of a microdialysis catheter according to an embodiment of the invention, with a carrier in the form of a multi-lumen tube.

One possible variant of a tubular carrier 1 with fluid channels is a multi-lumen tube 31. FIG. 3.1 shows a top view, FIG. 3.2 a longitudinal section (along A-A in FIG. 3.1) and FIG. 3.3 a transverse section (along B-B in FIG. 3.2) of an enlarged view of such a microdialysis catheter based on a multi-lumen tube.

The tubular outside wall 14 of the multi-lumen tube, i.e. a tube in which two lumina 16 with passage of fluid between them prevented by a dividing wall 15 are created, has side openings 17 in the area which, when the microdialysis catheter is in use, is inside the body, said openings allowing a connection for the passage of fluid to be made from the surrounding outer space 18 to the inner space 19 in the microdialysis catheter.

The dividing wall 15 between the two lumina 16 of the multi-lumen tube is, shortly before the distal end 10 of the microdialysis catheter, structured so that the two lumina 16 have connected fluid flows. The dividing wall 15 can, for example, starting from the front surface of the multi-lumen tube, be notched or removed using a suitable tool (such as a laser). One of the lumina 20 with connected fluid flow is used to supply a perfusion fluid. The other of the lumina 21 with connected third flow is used to remove the dialysate.

The multi-lumen tube structured in this way has a seal to prevent the passage of fluid from the surrounding outer space 18 at the distal end 10, in that the openings at the ends of the lumina are closed off with a seal 22.

The tube with the side openings 17 and the fluid connection 23 between the lumina 16 and the seal 22 at the distal end 10 is covered on the outside with a non-porous coating 13 which is diffusion-permeable to the analyte. The coating 13 covers the outside wall 14 of the tube in a defined area at the distal end 10 of the multi-lumen tube which, when the catheter is implanted, is in the tissue. The coating 13 is bonded to the outside wall 14. The side openings 17 introduced are covered by the coating 13, allowing the lumina 16 to be reached by the analyte.

The multi-lumen tube with the above-mentioned coating 13 may have another, biocompatible layer (not shown) through which the analyte can flow.

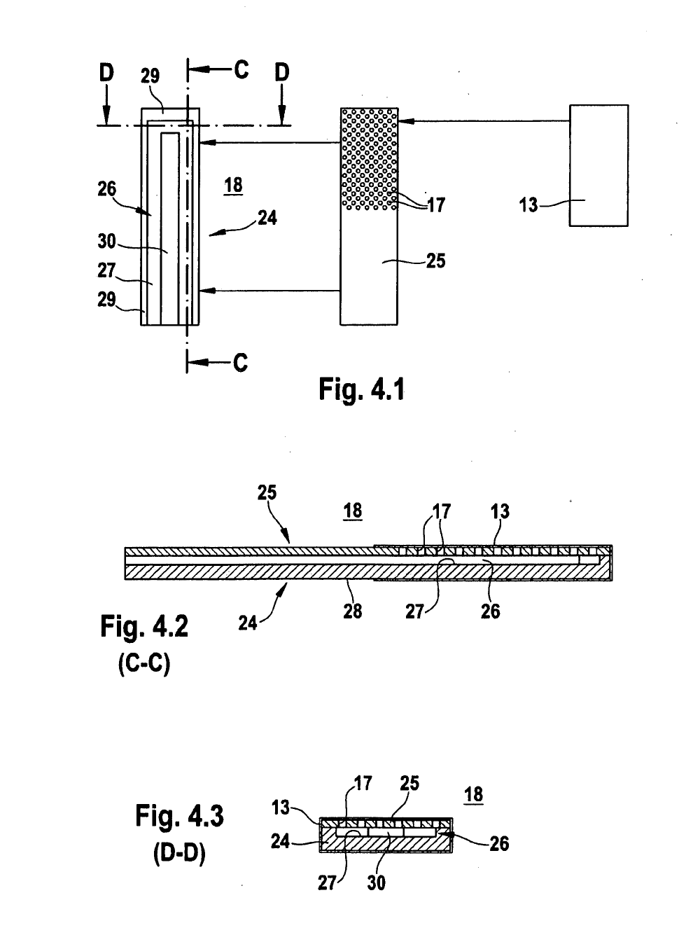

FIGS. 4.1 to 4.3 show schematically a fourth embodiment of a microdialysis catheter according to an embodiment of the invention with a carrier in the form of a flat substrate with a cover.

According to another embodiment of the present invention, the carrier of the microdialysis catheter is a flat substrate with a cover. FIG. 4.1 shows a top view of the individual layers in such a microdialysis catheter, FIG. 4.2 a longitudinal section (along C-C in FIG. 4.1) and FIG. 4.3 a transverse section (along D-D in FIG. 4.1) through such a microdialysis catheter based on a flat substrate 24 with a cover 25. The sections are shown enlarged in FIGS. 4.2 and 4.3.

The flat substrate 24 contains as channel a canal structure 26 for supplying a perfusion fluid and for removing a dialysate. The canal structure 26 is bounded by an outer wall 29 and an inner ridge 30 and closed by the cover 25 of the flat substrate 24, where the cover 25 has a large number of openings 17 which are covered by a non-porous coating 13 diffusion-permeable to an analyte and have a connection with the canal structure 26 for the passage of fluid.

To manufacture such a microdialysis catheter, a canal structure 26 is introduced into a flat substrate 24 allowing the supply of a perfusion fluid and the removal of the dialysate. The canal structure 26 is closed on all sides by the cover 25 of a groove 27 on the surface of the substrate 24 by a cover plate or cover foil. Openings 17 are made in the cover plate/foil 25, making a connection for the passage of fluid between the outer space 18 surrounding the microdialysis catheter and the canal structure 26.

The cover 25 containing the openings 17 receives on the outside a non-porous coating 13 which is diffusion-permeable to the analyte. The coating 13 covers the outside wall 28 of the substrate 24 and the cover 25. The openings 17 made are covered by the coating 13. The substrate 24 with the aforementioned coating 13 if necessary receives another, biocompatible coating (not shown) through which the analyte can flow.

Thus, embodiments of the microdialysis catheter and process for manufacturing the catheter are disclosed. One skilled in the art will appreciate that the teachings can be practiced with embodiments other than those disclosed. The disclosed embodiments are presented for purposes of illustration and not limitation, and the invention is only limited by the claims that follow.

What is claimed is:

1. A process for the manufacture of a microdialysis catheter, comprising:
providing a carrier containing at least one channel for the supply of a perfusion fluid and for the removal of a dialysate, the carrier having an area intended for implantation containing at least one opening that has a connection with the channel for the passage of fluid, and
coating the carrier with a non-porous coating that is firmly bonded to the carrier which is diffusion-permeable to an analyte, the coating externally at least partly covers the area intended for implantation and covers the opening.

2. The process as in claim 1 wherein the area intended for implantation of the carrier is coated with a polymer solution.

3. The process as in claim 2 wherein the carrier is coated by either dipping the carrier in a polymer solution, or spraying the carrier with the polymer solution, or squeegeeing the carrier with the polymer solution, or rotary coating the carrier with the polymer solution.

4. The process as in claim 2 wherein the carrier is coated with a polymer solution and the polymer solution is hardened by either heating, or irradiating, or electromagnetic radiation, or by water contact.

5. The process in claim 1 further comprising covering the coating with a layer of a biocompatible, porous material.

6. A microdialysis catheter, comprising:
a carrier containing at least one channel for supplying a perfusion fluid and for removing a dialysate, the carrier having an area intended for implantation;
at least one opening on the carrier that has a connection with the channel for the passage of fluid; and,
a non-porous coating externally bonded to the carrier, the non-porous coating is diffusion-permeable to an analyte and covers the area intended for implantation and the opening.

7. The microdialysis catheter in claim 6 wherein the coating is coated with a layer of a biocompatible material.

8. The microdialysis catheter in claim 6 wherein carrier comprises,
a tube having a distal end that is sealed and at least one side opening covered by a coating that is diffusion-permeable to an analyte;
a longitudinal inner space formed inside the tube, the longitudinal inner space containing at least one fluid channel for supplying a perfusion fluid and for removing a dialysate;
a connection in the fluid channel having at least one side opening for the passage of fluid.

9. The microdialysis catheter in claim 8 wherein the fluid channels in the inner space of the tube are lumina separated by a dividing wall with a passage for fluid in the dividing wall or two inner tubes with a connection for the passage of fluid between the two inner tubes.

10. The microdialysis catheter in claims 6 wherein the carrier comprises,
a flat substrate having a canal structure for supplying a perfusion fluid and for removing a dialysate; and,
a cover attached to the flat substrate enclosing the canal structure, the cover having at least one opening that is covered by a non-porous coating which is diffusion-permeable to an analyte and is connected with the canal structure for the diffusion of fluid from outside the non-porous coating through the opening.

11. A microdialysis catheter, comprising:
a carrier for providing mechanical stability having an external diameter in the range from about 0.5 mm to about 0.75 mm, the carrier containing at least one channel for supplying a perfusion fluid and for removing glucose and having an area intended for implantation, the carrier further comprising,
a tube having a distal end that is sealed and at least one side opening covered by a coating that is diffusion-permeable to an analyte,
a longitudinal inner space formed inside the tube, the longitudinal inner space containing at least one fluid channel for supplying a perfusion fluid and for removing a dialysate,
a connection in the fluid channel having at least one side opening for the passage of fluid,
at least one opening having an area from about 1 μm² to about 100 μm² on the carrier that has a connection with the channel for the passage of fluid; and,
a non-porous coating formed from a three dimensional polymer network externally bonded to the carrier, the non-porous coating is diffusion-permeable to glucose and covers the area intended for implantation and at least one opening.
